(19) United States
(12) Patent Application Publication
(10) Pub. No.: US 2018/0199644 A1
(43) Pub. Date: Jul. 19, 2018

(54) PLASTIC SURGICAL GLOVES WITH REINFORCED KNUCKLE AND PALM AREAS

(71) Applicant: ALBERT SNADO, BROOKLYN, NY (US)

(72) Inventor: ALBERT SANDO, BROOKLYN, NY (US)

(21) Appl. No.: 15/408,857

(22) Filed: Jan. 18, 2017

(51) Int. Cl. A41D 19/00; A61B 42/10

(52) U.S. Cl. CPC A41D 19/0096; A41D 2600/20; A61B 42/10

(57) ABSTRACT

The present invention provides a reinforced plastic glove having a coating that prevents piercing so that the glove can be used in the medical field, or in the food field. The surgical gloves are typically made of nitrile, latex, or vinyl. In addition, the reinforcing coating on these gloves can be either the same material as the glove, or the gloves can be coated with polyethylene or different plastics that would be most suitable for providing a reinforcement coating. The reinforcement coating is provided by coating the plastic glove on both the inner and outer surfaces, in the palm area, and in the knuckle area of the glove. The coating on the glove has a thickness in the range of 10 mm to 40 mm, and a width in the palm area of 90 mm on the surface of the glove. Also, the coating in the knuckle area is in the range of 10 mm wide and 50 mm long. The coating can be applied by a dipping process or a spraying process.

### FIELD OF THE INVENTION

The present invention relates to reinforced plastic surgical gloves, which are coated to withstand piercing which would make the gloves usable in the medical or food fields.

### BACKGROUND OF THE INVENTION

It is known in the prior art to provide disposable surgical gloves made of plastic material. However, such plastic gloves are subject to being punctured, torn, or ripped during use. This may occur when the person wearing the gloves is also wearing hand jewelry, which can puncture or tear the plastic glove to form holes. The present invention overcomes the problem with plastic gloves that can be pierced.

### OBJECTS OF THE INVENTION

It is an object of the present invention to provide a reinforced plastic surgical glove. Another object of the present invention is to provide a reinforced plastic surgical glove that can withstand punctures or tears.

Another object of the present invention is to provide reinforced knuckle and palm areas in the plastic surgical glove.

Another object of the present invention is to provide a reinforced plastic surgical glove which has an additional layer formed of polyethylene or other suitable plastic materials.

### SUMMARY OF THE INVENTION

The present invention provides a reinforced plastic glove having a coating that prevents piercing so that the glove can be used in the medical field, or in the food field. The surgical gloves are typically made of nitrile, latex, or vinyl. In addition, the reinforcing coating on these gloves can be either the same material as the glove, or the gloves can be coated with polyethylene or different plastics that would be most suitable for providing a reinforcement coating. The reinforcement coating is provided by coating the plastic glove on both the inner and outer surfaces, in the palm area, and in the knuckle area of the glove. The coating on the glove has a thickness in the range of 10 mm to 40 mm, and a width in the palm area of 90 mm on the surface of the glove. Also, the coating in the knuckle area is in the range of 10 mm wide and 50 mm long. The coating can be applied by a dipping process or a spraying process.

### BRIEF DESCRIPTION OF THE DRAWINGS

As shown in FIG. 1 is a perspective view of a plastic surgical glove having reinforced palm areas and knuckle areas; and

FIG. 2 is a perspective view of the back side of the plastic surgical glove having reinforced knuckle areas.

### DETAILED DESCRIPTION OF THE PREFERRED EMBODIMENT

The present invention provides a reinforced plastic glove having a plastic coating that prevents piercing, so that the reinforced glove can be used in the medical field, or in the food field. The surgical gloves are typically made of nitrile, latex, or vinyl. In addition, the reinforcing coating on these gloves can be either the same material as the glove, or the gloves can be coated with polyethylene or different plastics that would be most suitable for providing a reinforcement coating. The reinforcement coating is provided by coating the plastic glove on both the inner and outer surfaces, in the palm area 18, and in the knuckle areas 16a of the glove, on both the inner and outer surfaces.

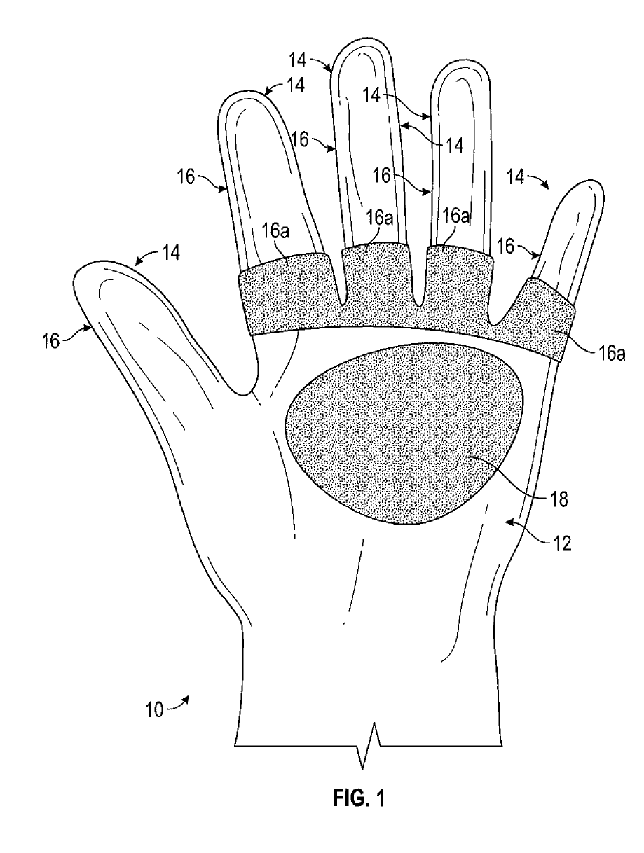

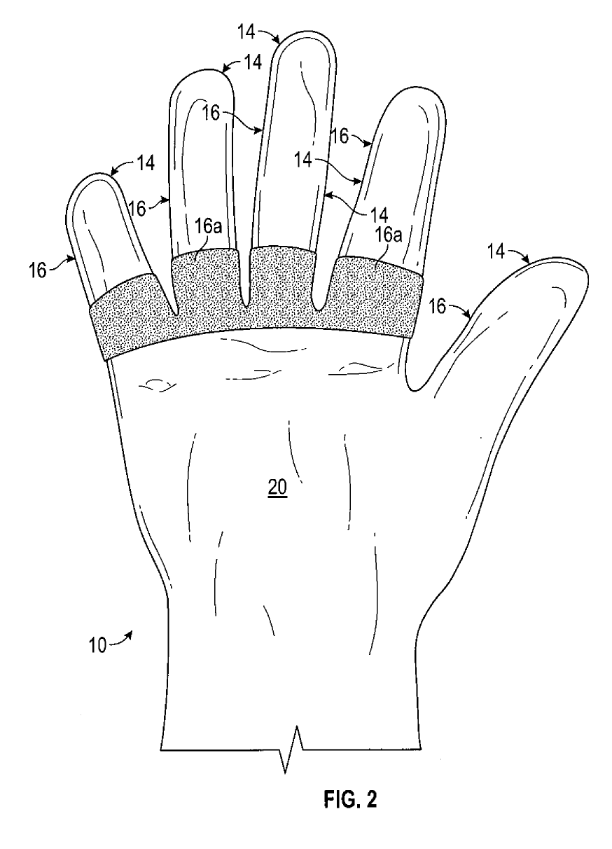

As shown in FIG. 1, the present invention provides a surgical glove 10 formed of plastic material such as polyethylene or different plastics. On the inner surface 12 of glove 10, there is a palm section 18 and finger sections 14. FIG. 2 shows the outer knuckle areas 16, and an outer surface 20.

An additional layer of reinforced plastic is provided to form reinforced knuckle sections 16a and reinforced palm section 18.

The reinforcement is provided by coating the plastic glove on both the inside and outside surfaces, and in the palm area, and in the knuckle area of the glove. The coating of the glove has a thickness in the range of 10 mm to 40 mm, and a width of 90 mm in the palm area, and the coating in the finger area is 10 mm wide and 50 mm long.

The coating can be applied by a dipping process or a spraying process to apply polyethylene reinforcement coating or other suitable reinforced plastic material as an additional coating layer.

### ADVANTAGES OF THE PRESENT INVENTION

An advantage of the present invention is to provide a reinforced plastic surgical glove.

Another advantage of the present invention is to provide a reinforced plastic surgical glove that can withstand punctures or tears.

Another advantage of the present invention is to provide reinforced knuckle and palm areas in the plastic surgical glove.

Another advantage of the present invention is to provide a reinforced plastic surgical glove which has an additional layer formed of polyethylene or other suitable reinforced plastic materials.

A latitude of modification, change and substitution is intended in the foregoing disclosure, and in some instances, some features of the invention will be employed without a corresponding use of other features. Accordingly, it is appropriate that the appended claims be construed broadly and in a manner consistent with the spirit and scope of the invention herein.

What is claimed is:

1) A reinforced plastic glove having a palm area and finger areas that prevents piercing so that the glove can be used in the medical field, or in the food field, wherein the glove is formed of nitrile, latex, or vinyl, comprising:
(a) a reinforcing material is provided by coating said plastic glove on both the inside and outside surfaces thereof with a coating of polyethylene or other suitable plastic material;
(b) said palm area 18 and said knuckle areas 16a of the glove are coated with a layer of polyethylene or other suitable plastic material;
(c) said reinforcing coating of the glove has a thickness in the range of 10 mm to 40 mm;
(d) said palm area having a coating width of 90 mm, and said coating in the finger area is 10 mm wide and 50 mm long; and
(e) said coating is formed of polyethylene or other suitable plastic materials, which is applied by a dipping process or a spraying process in the surfaces of the glove.
